(19) United States
(12) Patent Application Publication
(10) Pub. No.: US 2002/0110564 A1
(43) Pub. Date: Aug. 15, 2002

(54) Nutriceutical composition and methods

(76) Inventor: Mark Itokawa, Gardena, CA (US)

Correspondence Address: CHRISTIE, PARKER & HALE, LLP, P.O. Box 7068, Pasadena, CA, 91109-7068, US

(21) Appl. No.: 09/733,745

(22) Filed: Dec. 8, 2000

Related U.S. Application Data
(60) Provisional application No. 60/170,189, filed on Dec. 10, 1999.

(51) Int. Cl. A61K 35/84

(52) U.S. Cl. 424/195.15

(57) ABSTRACT

Nutriceutical compositions prepared from the basidiomycete Agaricus blazei Murill, grown in China and more particularly from the Fujian province of China, are provided. The invention also provides methods for using such compositions to prevent and treat cancer, and to lower blood sugar levels.

### FIELD OF THE INVENTION

This invention relates to nutriceutical compositions prepared from the basidiomycete Agaricus blazei Murill, grown in China and more particularly from the Fujian province of China, and to the methods of use of such compositions to prevent and treat cancer, and to lower blood sugar levels.

### BACKGROUND OF THE INVENTION

The Agaricus blazei Murill mushroom (hereinafter, ABM) is a basidiomycete which grows wild in the fields and mountains of the Sao Jose do Rio Preto district, located northwest of Sao Paulo, Brazil. Researchers noted some thirty years ago that inhabitants of this region had a low incidence of certain adult diseases. This was attributed to ABM, which was widely used in the local diet.

ABM was subsequently introduced into Japan. Researchers there found that a polysaccharide-protein complex isolated from cultured mycelia of ABM was an effective in vivo antitumor agent against four kinds of established mouse tumors: Sarcoma 180, Ehrlich ascites carcinoma, Shionogi carcinoma 42 and Meth A fibrosarcoma. (H. Ito, K. Shimura, H. Itoh, M. Kawade (January 1997) Antitumor effects of a new polysaccharide-protein complex (ATOM) prepared from Agaricus blazei (Iwade strain 101) “Himematsutake” and its mechanisms in tumor-bearing mice, Anticancer Research 17(1A):227-284.

Subsequently, researchers identified a polysaccharide-containing fraction in extracts prepared from ABM containing an alpha-1,4-glucan-beta-1,6-glucan complex, which has a selective cytotoxic effect on tumor cells in vitro while having no effect on normal cells. When used in vivo, this complex produces a marked inhibition of Meth A tumors. Mice receiving injections of the ABM polysaccharide complex also have elevated serum levels of immunosuppressive acidic protein, suggesting the possible activation of granulocytes. (Y. Fujiyama, Y. Suzuki, R. Katakura, and T. Ebina, (January-February 1999) Tumor-specific cytocidal and immunopotentiating effects of relatively low molecular weight products derived from the basidiomycete Agaricus blazei Murill, Anticancer Research 19(1A):113-18.)

ABM has thus become popular, particularly in Japan and Brazil, as a food supplement for its health benefits. (Hereinafter, ABM grown in Brazil shall be referred to as ABM—Brazil while ABM grown in Japan shall be referred to as ABM—Japan). However, it would be advantageous to find ABM with enhanced anti-cancer activity and which confers other health benefits, such as lowering blood sugar levels.

### SUMMARY OF THE INVENTION

The present invention is directed to compositions and methods for treating and/or preventing cancer and to compositions and methods for lowering blood sugar levels.

### DETAILED DESCRIPTION OF THE INVENTION

The nutriceutical composition of the present invention comprises ABM grown in China (hereinafter, ABM—China), and particularly in the Fujian province of China. These mushrooms are grown either in the wild or are cultivated. Once harvested, the mushrooms are dried and used to make a “tea” or are ground up and encapsulated for use as a supplement. The dosage of dried ABM—China is selected such that the concentration of ABM—China in the human receiving the formulation will be at a non-toxic, but therapeutically effective, concentration in the host.

Surprisingly, it has been found that ABM—China has an enhanced natural killer (NK) cell activity relative to ABM—Brazil and ABM—Japan. NK cells are small lymphocyte-like cells that spontaneously kill a variety of tumor cells in culture. The enhanced NK activity of ABM—China is most pronounced on non-adherent cancer cells, which is significant as diminished adherence may contribute to metastatic spread of the cancer.

It has also surprisingly been found that extracts from ABM—China lower blood sugar levels in vitro over a seven-day period, relative to extracts prepared from ABM—Brazil or ABM—Japan.

The concentration of antioxidants in ABM—China relative to ABM—Japan and ABM—Brazil was also examined. Antioxidants are known to reduce the level of free radicals in the body. Free radicals are highly reactive molecules generated during normal cell metabolism by biochemical redox reactions. Free radicals also result from exposure to environment hazards such as ultraviolet light, gamma radiation, cigarette smoke, and environmental pollutants. These free radicals can attack and damage cell structures in the body and have been implicated in diseases such as cancer, atherosclerosis, rheumatoid arthritis, and diabetes.

ABM—China is dried after harvesting, preferably by natural air drying, optionally with the application of heat. Although the ABM—China can also be freeze-dried, it has been found that the freeze-dried product provides fewer health benefits than the naturally dried product.

ABM—China is administered orally and may be dispersed in a pharmaceutically acceptable carrier. For example, it can be in tablet or capsule carriers, which can include components such as excipients, bulking agents, lubricants, disintegrants, solubilizing solvents, dyes and the like. It can also be administered in a suitable liquid carrier, as, for example, water.

In a further exemplary embodiment of the invention, a material such as a cellulosic web or other mesh product permeable to water can be used as a bag to contain dried ABM—China. The bag containing dried ABM—China is submerged in a suitable liquid carrier, such as water, and allowed to soak for an amount of time sufficient to extract the active nutriceutical ingredients from the dried ABM—China.

In one exemplary embodiment of the invention, powdered dried ABM—China is provided in a capsule form as is known in the art. In one embodiment, a therapeutically effective dose of from about 150 mg to about 24 gm of powdered dried ABM—China is administered per day to the human or animal host. Higher doses of powdered dried ABM—China may be administered, depending on economic and taste considerations.

In another exemplary embodiment of the invention, dried ABM—China is provided in an aqueous solution. To make the aqueous solution, dried ABM—China is placed in a container with cold water and soaked from about five hours to about 24 hours, preferably in a refrigerator. The ABM—China is transferred from the cold water, which is retained, to a container with hot water, and is boiled from about 8 to about 15 minutes. The ABM—China is then removed, and the cold water and the boiled water are combined to form a tea which is orally administered about one to about three times per day, preferably before meals. The volumes of water used to soak and to boil the ABM—China may be varied according to the desired strength of the resulting tea. Typically, about 2 to about 5 cups of cold water and between about 2 to about 5 cups of hot water are used with 10 g ABM—China.

### EXAMPLE 1

### Administration to Reduce the Risk of Cancer

An exemplary embodiment for reducing the risk of cancer in accordance with practice of principles of this invention comprises orally administering capsules containing a therapeutically effective dosage of dried ABM—China to the host. The minimum therapeutically effective dosage of ABM—China varies, depending on the weight of the host, from about 150 mg to about 1.5 gm of ABM—China per day, as provided in Table 1. Higher doses of ABM—China may be administered if desired, subject to economic and taste considerations.

TABLE 1

| Weight of Host | Minimum Amount of ABM-China |
|---|---|
| 50 kg/110 lb | 150 mg-1 gm |
| 60 kg/132 lb | 180 mg-1 gm |
| 70 kg/154 lb | 210 mg-1 gm |
| 80 kg/176 lb | 240 mg-1 gm |
| 90 kg/198 lb | 270 mg-1.5 gm |
| 100 kg/220 lb | 300 mg-1.5 gm |
| 110 kg/242 lb | 330 mg-1.5 gm |
| 120+ kg/264+ lb | 360 mg-1.5 gm |

### EXAMPLE 2

### Administration to Treat Cancer

An exemplary embodiment for treating cancer in accordance with practice of principles of this invention comprises orally administering capsules containing a therapeutically effective dosage of dried ABM—China to the host. The minimum therapeutically effective dosage of ABM—China administered varies, depending on the weight of the host and the stage of the cancer, from about 300 mg to about 24.0 gm of ABM—China per day, as provided in Table 2. Higher doses of ABM—China may be administered, depending on cost and taste considerations.

TABLE 2

| | Minimum Amount of | |
|---|---|---|
| | ABM-China for | Minimum Amount of ABM- |
| | Stage one/two | China for Stage |
| Weight of Host | Cancer | three/four Cancer |
| 50 kg/110 lb | 300 mg-6 gm | 500 mg-10 gm |
| 60 kg/132 lb | 360 mg-8 gm | 600 mg-12 gm |
| 70 kg/154 lb | 420 mg-10 gm | 700 mg-16 gm |
| 80 kg/176 lb | 480 mg-10 gm | 800 mg-20 gm |
| 90 kg/198 lb | 540 mg-12 gm | 900 mg-20 gm |
| 100 kg/220 lb | 600 mg-12 gm | 1000 mg-22 gm |
| 110 kg/242 lb | 660 mg-14 gm | 1100 mg-24 gm |
| 120+ kg/264+ lb | 720 mg-16 gm | 1200 mg-24 gm |

### EXAMPLE 3

### ABM—China Tea Used To Prevent and To Treat Cancer

An exemplary embodiment for preventing and treating cancer in accordance with practice of principles of this invention comprises orally administering a liquid tea containing a therapeutically effective dosage of ABM—China to the host.

The tea is made by placing 10 gm of dried ABM—China into a container with four cups of cold water. The ABM—China is allowed to soak for about five hours in a refrigerator. The ABM—China is then removed, retaining the cold water in which it was soaked. The ABM—China is added to two cups of hot water and allowed to boil from about 8 to about 10 minutes. After boiling, the ABM—China is removed and the boiled water is added to the retained cold water to produce a tea which is stored in a refrigerator.

The tea is administered orally once a day in a therapeutically effective dosage as provided: For preventing cancer or for treating stage one cancer, about one-fifth of the tea produced is administered. For treating stage two cancer, about two-fifths of the tea produced is administered. For treating stage three cancer, about four-fifths of the tea produced is administered. For treating stage four cancer, all of the tea is administered.

In general practice, the exact volume of water used in preparing the tea is not critical. Rather, it is the amount of extracted ABM—China that is important. In example 3, the tea is prepared with 10 g of ABM—China and a total of 6 cups of water. For use in reducing the risk of cancer, for example, one fifth of this 6-cup total volume, or 1.2 cups, is administered daily. However, the amount of water may be varied to taste, so long as the proportionate amount of the total volume is administered. Thus, for example, the 10 g of ABM—China can be prepared using a total of 8 cups water. In this case, 1.6 cups, or one fifth of the 8-cup total volume, is administered daily to reduce the risk of cancer.

Similarly, the amount of ABM—China used to make the tea may be varied, so long as the amount used is sufficient for its intended therapeutic purpose, as follows: For reducing the risk of cancer or for treating stage one cancer, administer daily a tea prepared using at least about 2 grams ABM—China; for treating stage two cancer, administer daily a tea prepared using at least about 4 grams ABM—China; for treating stage three cancer, administer daily a tea prepared using at least about 8 grams ABM—China; and for treating stage four cancer, administer daily a tea prepared using at least about 10 grams of ABM—China. Higher amounts of ABM—China may be used in preparing the tea, depending on cost and taste considerations.

Thus, for example, a tea for use in preventing cancer or for treating stage one cancer may be prepared from 10 grams ABM—China, in which case at least about one-fifth of the final volume of tea is administered each day regardless of the final volume. Alternatively, the tea may be prepared using at least about 2 grams ABM—China, in which case the entire final volume of tea is administered each day.

A tea for use in treating stage two cancer may be prepared from 10 grams ABM—China, in which case at least about two-fifths of the final volume of tea is administered each day regardless of the final volume. Alternatively, the tea may be prepared using at least about 4 grams ABM—China, in which case the entire final volume of tea is administered each day.

A tea for use in treating stage three cancer may be prepared from 10 grams ABM—China, in which case at least about four-fifths of the final volume of tea is administered each day regardless of the final volume. Alternatively, the tea may be prepared using at least about 8 grams ABM—China, in which case the entire final volume of tea is administered each day.

And finally, a tea for use in treating stage four cancer may be prepared from at least about 10 grams ABM—China, in which case the entire final volume of tea is administered each day. Other variations are apparent.

The following experiments relate to in vitro tests to determine the effect of extracts from ABM—China on NK-cell function. Specifically, these experiments relate to the effect of extracts from ABM—China on the rate at which NK-cells kill cells from a human chronic myelogenous leukemia cell line relative to extracts prepared from ABM—Brazil or ABM—Japan.

### EXAMPLE 4

### Effect of ABM—China on NK-cell Function in Vitro

In this example, NK-cell activity was assessed using a variation of standard cytotoxicity assays. Cells from K562, a human chronic myelogenous leukemia cell line, maintained in complete RMPI medium and 10% fetal bovine serum (FBS) (Sigma, St. Louis, Mo.), were used as target cells. The K562 cells were labeled with 20 μC ⁵¹Cr (ICN, Costa Mesa, Calif.) for 1 hour at 37° C. in 5% CO₂, washed four times with medium, and cell suspensions were pipetted at a concentration of 5×10³ cells/well into 96-well U-bottom microtiter plates. Effector cells (peripheral blood mononuclear cells [PBMC] isolated from the blood by sucrose density gradient centrifugation) were added in triplicate to the wells at an effector:target ratio of 20:1, followed by flour (negative control), adriamycin (positive control) or one of the three mushroom extracts prepared from ABM—China, ABM—Japan, or ABM—Brazil, each at a final concentration of 200 μg/ml. Control wells without effectors contained target cells and mushroom extract alone for determination of spontaneous lysis, or 3% Triton X-100 for evaluation of total lysis. After incubation for 4 hours at 37° C. in 5% CO₂, the plates were centrifuged for 10 minutes at 1,500×g, and 50 μl of cells removed, stained with a 1:10 dilution of trypan blue, and a random field assessed for dead cells and live cells in 100 randomly-chosen cells. The results are shown in Table 3.

TABLE 3

| | # of Live | # of Dead | Mean of Dead | % increase |
|---|---|---|---|---|
| Sample Name | K562 Cells | K562 Cells | Cells | in killing |
| Flour-a | 91 | 9 | 8.3 | |
| Flour-b | 93 | 7 | | |
| Flour-c | 91 | 9 | | |
| Positive-a | 80 | 20 | 26.3 | |
| Positive-b | 76 | 24 | | |
| Positive-c | 65 | 35 | | |
| ABM-China-a | 90 | 10 | 16.0 | 93% |
| ABM-China-b | 77 | 23 | | |
| ABM-China-c | 85 | 15 | | |
| ABM-Japan-a | 80 | 20 | 15.7 | 89.10% |
| ABM-Japan-b | 86 | 14 | | |
| ABM-Japan-c | 87 | 13 | | |
| ABM-Brazil-a | 84 | 16 | 13.7 | 65.10% |
| ABM-Brazil-b | 87 | 13 | | |
| ABM-Brazil-c | 88 | 12 | | |

In table 3, the negative control, flour, resulted in only a means of 8.3 dead target cells. Extracts from ABM increased the number of dead target cells over that of flour by between 65.1% and 93%. The highest increase in number of dead target cells is seen for extracts prepared from ABM—China.

### EXAMPLE 5

### Effect of ABM—China on NK-function in Vitro, Comparing Adherent Cells to Non-adherent Cells

In this example, the experiment of Example 4 was repeated with PBMC effector cells in which adherent cells in the PBMC population were separated from non-adherent cells following incubation at 37° C. in 5% CO₂ and which the adherent cells and non-adherent cells were assessed separately for NK activity. The results for adherent cells are shown in Table 4. The results for non-adherent cells is shown in Table 5.

TABLE 4
Adherent Cells

| | # of Live | # of Dead | Mean of Dead | % increase |
|---|---|---|---|---|
| Sample Name | K562 Cells | K562 Cells | Cells | in killing |
| Flour-a | 62 | 38 | 35.7 | |
| Flour-b | 61 | 39 | | |
| Flour-c | 70 | 30 | | |
| Positive-a | 42 | 58 | 57.3 | |
| Positive-b | 46 | 54 | | |
| Positive-c | 40 | 60 | | |
| ABM-China-a | 53 | 47 | 44.0 | 23% |
| ABM-China-b | 54 | 46 | | |
| ABM-China-c | 61 | 39 | | |
| ABM-Japan-a | 53 | 47 | 46.0 | 29% |
| AEM-Japan-b | 59 | 41 | | |
| ABM-Japan-c | 50 | 50 | | |
| ABM-Brazil-a | 61 | 39 | 42.3 | 19% |
| ABM-Brazil-b | 54 | 46 | | |
| ABM-Brazil-c | 58 | 42 | | |

TABLE 5
Non-Adherent Cells

| | # of Live | # of Dead | Mean of Dead | % increase |
|---|---|---|---|---|
| Sample Name | K562 Cells | K562 Cells | Cells | in killing |
| Flour-a | 85 | 15 | 14.0 | |
| Flour-b | 89 | 11 | | |
| Flour-c | 84 | 16 | | |
| Positive-a | 55 | 45 | 38.0 | |
| Positive-b | 68 | 32 | | |
| Positive-c | 63 | 37 | | |
| ABM-China-a | 78 | 22 | 21.0 | 20% |
| ABM-China-b | 77 | 23 | | |
| ABM-China-c | 82 | 18 | | |
| ABM-Japan-a | 78 | 22 | 17.3 | 24% |
| ABM-Japan-b | 86 | 14 | | |
| ABM-Japan-c | 84 | 16 | | |
| ABM-Brazil-a | 87 | 13 | 13.7 | <2% |
| ABM-Brazil-b | 82 | 18 | | |
| ABM-Brazil-c | 90 | 10 | | |

As can be seen in Table 5, the maximum increase in killing of non-adherent cells, 50%, results from the addition of extracts from ABM—China. By contrast, extracts from ABM—Japan produces only a 24% increase in killing while ABM—Brazil produces less than a 2% increase in killing of non-adherent cells.

### EXAMPLE 6

### Administration to Reduce Blood Sugar Levels

An exemplary embodiment for reducing blood sugar levels in accordance with practice of principles of this invention comprises administering a minimum therapeutic dosage from about 150 mg to about 24.0 gm of ABM—China to the host per day. Higher dosages of ABM—China may be administered, depending on taste and cost considerations.

The following experiment relates to in vitro tests to determine the effect of ABM—China on glucose levels in blood over a 7 day period.

### EXAMPLE 7

### Effect of ABM—China on Blood Glucose Levels

In this experiment, baseline plasma blood glucose (BG) levels were measured at baseline from a normal control, then at 24 hours and at 7 days using a glucometer by the glucose oxidase method. Flour, known to lower BG levels by a small percentage (8+/−1.1%) was used as a positive control; no additive as a negative control. Three samples each of extracts of ABM—China, ABM—Japan, and ABM—Brazil, were added to a final concentration of 200 μg/ml. The flour contained 4% calcium, 8% iron, 10% Thiamine, 6% riboflavin and 8% niacin. The results are shown in Table 6.

TABLE 6

| Sample | Baseline BG | Mean Baseline BG | BG @ 24 hr | Mean BG @ 24 hr | BG @ day 7 | Mean BG @ day 7 | Decrease in BG |
|---|---|---|---|---|---|---|---|
| Units | mg/dl | mg/dl | mg/dl | mg/dl | mg/dl | mg/dl | % |
| Plasma - a | 86 | 86 | 87 | 87.3 | 86 | 85.3 | |
| Plasma - b | 86 | | 88 | | 84 | | |
| Plasma - c | 87 | | 87 | | 86.0 | | |
| Flour - a | N/A | N/A | 84 | 81.7 | 80 | 79.3 | 9.2 |
| Flour - b | N/A | | 79 | | 79 | | |
| Flour - c | N/A | | 82 | | 79.0 | | |
| ABM - | N/A | N/A | 79 | 80.3 | 78 | 77.7 | 11.1 |
| China - a | | | | | | | |
| ABM - | N/A | | 80 | | 79 | | |
| China - b | | | | | | | |
| ABM - | N/A | | 82 | | 76.0 | | |
| China - c | | | | | | | |
| ABM - | N/A | N/A | 83 | 81.7 | 79 | 80.0 | 8.4 |
| Japan - a | | | | | | | |
| ABM - | N/A | | 82 | | 80 | | |
| Japan - b | | | | | | | |
| ABM - | N/A | | 80 | | 81.0 | | |
| Japan - c | | | | | | | |
| ABM - | N/A | N/A | 79 | 82.0 | 80 | 79.7 | 8.8 |
| Brazil - a | | | | | | | |
| ABM - | N/A | | 83 | | 80 | | |
| Brazil - b | | | | | | | |
| ABM - | N/A | | 84 | | 79.0 | | |
| Brazil - c | | | | | | | |

As expected, flour produced a decrease in BG levels of approximately 9.2%. The greatest decrease in BG levels, 11.1%, resulted from the addition of extracts prepared from ABM—China. By contrast, extracts prepared from ABM—Japan or ABM—Brazil produced an 8.4% and 8.8% decrease in BG levels, respectively.

### EXAMPLE 8

### Toxicity Assay for ABM—China

In this experiment, PBMC from a normal control were suspended into wells of 96-well U-bottom microtiter plates followed by two 10-fold increasing concentrations of ABM extracts from 200 μg/ml to 2000 μg/ml. After incubation at 37° C. in 5% CO₂ for 4 hours, 10 μl of the suspension was removed, diluted 1:10 with trypan blue, and 100 PBMC assessed for viability in a random field. The results are shown in Table 7.

TABLE 7

| Sample | Result |
|---|---|
| ABM-China (2 mg) | No toxicity observed |
| ABM-China (200 μg) | No toxicity observed |
| ABM-Japan (2 mg) | No toxicity observed |
| ABM-Japan (200 μg) | No toxicity observed |
| ABM-Brazil (2 mg) | No toxicity observed |
| ABM-Brazil (200 μg) | No toxicity observed |

No toxicity was observed at concentrations of ABM—China up to 2 mg/ml, which exceeds the maximum concentration which can be achieved in human blood plasma.

### EXAMPLE 9

### Anti-Oxidant Assay

This experiment was performed using a Total Antioxidant Status Assay Kit sold by Calbiochem-Novabiochem Corp., Calbiochem Cat. No. 615700 (La Jolla, Calif.). The kit was used to assess the antioxidant potential of solubilized ABM—China. The assay was performed in accordance with the directions provided in the kit.

This assay works by utilizing the ability of antioxidants in a sample to inhibit the oxidation of ABTS (2,2′-Azino-di-[3-ethylbenzthiazoline sulphonate]) to ABTS^{•+} by metmyoglobin (a peroxidase). The amount of ABTS^{•+} produced is monitored by reading the absorbance at 600 nm. Under the conditions of the assay, antioxidants in the sample suppress absorbance at 600 NM in proportion to their concentration.

To perform the assay, the spectrophotometer is first zeroed at 600 NM against air with a set temperature of 37° C. The assay is initiated by adding 20 μl of either water (blank), 1.5 mM 6-Hydroxy-2,5,7,8-tetramethylchroman-2-carboxylic acid (standard), or the sample to be tested (in this case, solubilized ABM), to 1 ml of chromogen (metmyoglobin and ABTS) in cuvettes. The cuvettes are mixed and the initial absorbance read (Ao). Next, 200 μl of substrate (stabilized H₂O₂) is added. After exactly 3 minutes, the absorbance at 600 NM is again read (A). The antioxidant concentration can be calculated using the following formulae:

ΔA=A−Ao

Antioxidant Concentration (mM)=

$$Antioxidant Concentration ( mM ) = \frac{1.5 mM ( Δ A Blank - Δ A Sample}{( Δ A Blank - Δ A Standard )}$$

If the antioxidant concentration is greater than 2.5 mM, it is necessary to dilute the sample with 0.9% NaCl and re-assay.

This antioxidant assay was used on solubilized 200 μg samples of ABM. Each sample was tested twice and the results averaged. The results are shown in Table 8.

TABLE 8

| | | | | Mean | Antioxidant |
|---|---|---|---|---|---|
| Sample | Ao | A | ΔA | ΔA | Concentration |
| Blank | 0.000 | 0.143 | 0.143 | | |
| Standard | 0.001 | 0.004 | 0.003 | | |
| Flour | 0.000 | 0.138 | 0.138 | 0.1385 | 0.0482 mM |
| Flour | 0.000 | 0.139 | 0.139 | | |
| ABM - China | 0.000 | 0.139 | 0.139 | 0.141 | 0.0214 mM |
| ABM - China | 0.000 | 0.143 | 0.143 | | |
| ABM - Japan | 0.000 | 0.141 | 0.141 | 0.1415 | 0.0161 mM |
| ABM - Japan | 0.000 | 0.142 | 0.142 | | |
| ABM - Brazil | 0.000 | 0.141 | 0.141 | 0.1415 | 0.0161 mM |
| ABM - Brazil | 0.000 | 0.142 | 0.142 | | |

The above descriptions of exemplary embodiments of the methods and compositions of the present invention are for illustrative purposes. Because of variations, which will be apparent to those skilled in the art, the present invention is not intended to be limited to the particular embodiments described above.

What is claimed is:

1. A nutriceutical composition, comprising ABM—China.

2. The composition of claim 1, wherein the ABM—China is in solid dosage form.

3. The composition of claim 1, wherein the ABM—China is in an aqueous solution.

4. The composition of claim 3, wherein the aqueous solution is made by the steps of soaking the ABM—China in cold water for at least about five hours, transferring the ABM—China to hot water, boiling the ABM—China, and combining the boiled water with the cold water.

5. A composition for reducing the risk of cancer in humans, comprising ABM—China.

6. The composition of claim 5, wherein the ABM—China is in solid dosage form.

7. The composition of claim 5, wherein the ABM—China is in an aqueous solution.

8. The composition of claim 7, wherein the aqueous solution is made by the steps of soaking a therapeutically effective amount of ABM—China in cold water for at least about five hours, transferring the ABM—China to hot water, boiling the ABM—China for about 8 to about 15 minutes, and combining the boiled water with the cold water.

9. The composition of claim 8, wherein the therapeutically effective amount of ABM—China is from about 2 grams to about 10 grams.

10. The composition of claim 8, wherein the therapeutically effective amount of ABM—China is at least about 2 grams.

11. A method for reducing the risk of cancer in humans, comprising administering to a person in need thereof a therapeutically effective amount of ABM—China.

12. The method of claim 11, wherein the therapeutically effective amount of ABM—China is provided in a solid dosage form.

13. The method of claim 12, wherein the therapeutically effective amount of ABM—China is from about 150 mg to about 1.5 gm per day.

14. The method of claim 12, wherein the therapeutically effective amount of ABM—China is at least about 150 mg per day.

15. The method of claim 11, wherein the therapeutically effective amount of ABM—China is administered at least once a day in an aqueous solution.

16. The method of claim 15, wherein the aqueous solution is made by the steps of soaking at least about 2 grams of ABM—China in cold water for at least about five hours, transferring the ABM—China to hot water, boiling the ABM—China for about 8 to about 15 minutes, and combining the boiled water with the cold water.

17. A composition for treating cancer in humans, comprising ABM—China.

18. The composition of claim 17, wherein the ABM—China is in solid dosage form.

19. The composition of claim 17, wherein the ABM—China is in an aqueous solution.

20. The composition of claim 19, wherein the aqueous solution is made by the steps of soaking a therapeutically effective amount of ABM—China in cold water for at least about five hours, transferring the ABM—China to hot water, boiling the ABM—China for about 8 to about 15 minutes, and combining the boiled water with the cold water.

21. The composition of claim 20, wherein the therapeutically effective amount of ABM—China is from about 2 grams to about 10 grams.

22. The composition of claim 20, wherein the therapeutically effective amount of ABM—China is at least about 2 grams.

23. A method for treating cancer in humans, comprising administering to a person in need thereof a therapeutically effective amount of ABM—China.

24. The method of claim 23, wherein the therapeutically effective amount of ABM—China is provided in a solid dosage form.

25. The method of claim 24, wherein the therapeutically effective amount of ABM—China is from about 300 mg to about 24 gm per day.

26. The method of claim 24, wherein the therapeutically effective amount of ABM—China is at least about 300 mg.

27. The method of claim 23, wherein the therapeutically effective amount of ABM—China is administered at least once a day in an aqueous solution.

28. The method of claim 27, wherein the aqueous solution is made by the steps of soaking from about 2 grams to about 10 grams of ABM—China in cold water for at least about five hours, transferring the ABM—China to hot water, boiling the ABM—China for about 8 to about 15 minutes, and combining the boiled water with the cold water.

29. The method of claim 27, wherein the aqueous solution is made by the steps of soaking at least about 2 grams of ABM—China in cold water for at least about five hours, transferring the ABM—China to hot water, boiling the ABM—China for about 8 to about 15 minutes, and combining the boiled water with the cold water.

30. A composition for lowering blood sugar levels in humans, comprising ABM—China.

31. The composition of claim 30, wherein the ABM—China is in solid dosage form.

32. The composition of claim 30, wherein the ABM—China is in an aqueous solution.

33. The composition of claim 32, wherein the aqueous solution is made by the steps of soaking a therapeutically effective amount of ABM—China in cold water for at least about five hours, transferring the ABM—China to hot water, boiling the ABM—China for about 8 to about 15 minutes, and combining the boiled water with the cold water.

34. The composition of claim 33, wherein the therapeutically effective amount of ABM—China is from about 150 milligrams to about 24 grams.

35. A method for lowering blood sugar levels in humans, comprising administering to a person in need thereof a therapeutically effective amount of ABM—China.

36. The method of claim 35, wherein the therapeutically effective amount of ABM—China is provided in a solid dosage form.

37. The method of claim 35, wherein the therapeutically effective amount of ABM—China is administered at least once a day in an aqueous solution.

38. The method of claim 37, wherein the aqueous solution is made by the steps of soaking from about 150 milligrams to about 24 grams of ABM—China in cold water for at least about five hours, transferring the ABM—China to hot water, boiling the ABM—China for about 8 to about 15 minutes, and combining the boiled water with the cold water.

39. The method of claim 35, wherein the therapeutically effective amount of ABM—China is from about 150 mg to about 24 gm per day.

40. The method of claim 35, wherein the therapeutically effective amount of ABM—China is at least about 150 mg per day.
